(19) United States
(12) Patent Application Publication
(10) Pub. No.: US 2026/0076820 A1
(43) Pub. Date: Mar. 19, 2026

(54) APPARATUS WITH COMPRESSIBLE CASING FOR RECEIVING DISCHARGED URINE

(71) Applicant: PureWick Corporation, Covington, GA (US)

(72) Inventors: Kathleen Davis, Atlanta, GA (US); Katie Grupp, Alpharetta, GA (US)

(21) Appl. No.: 19/335,722

(22) Filed: Sep. 22, 2025

Related U.S. Application Data
(63) Continuation of application No. 17/451,345, filed on Oct. 19, 2021, now Pat. No. 12,440,370.
(60) Provisional application No. 63/094,498, filed on Oct. 21, 2020.

(51) Int. Cl. A61F 5/455; A61F 5/44; A61G 9/00; A61M 1/00

(52) U.S. Cl. CPC A61F 5/455; A61F 5/4404; A61G 9/006; A61M 1/87

(57) ABSTRACT

A device suitable for collecting and transporting urine away from the body of a person including a casing defining an internal volume and an opening. The casing can include a compressible material which is elastically deformable when the device is disposed in a pubic region of a female user. The device can include a porous material disposed within the internal volume and in fluid communication with the opening. The porous material can wick, draw, or direct fluid, such as urine, away from the pubic region of the female user and into the internal volume. The device can include a fluid conduit disposed within the internal volume and enabling extraction of fluid from the internal volume. The device can include a fluid impermeable housing disposed within the internal volume and adhered or otherwise affixed to the casing.

### CROSS-REFERENCE TO RELATED APPLICATIONS

This application is a continuation of U.S. application Ser. No. 17/451,345 filed on Oct. 19, 2021, which claims priority to U.S. Provisional Patent Application No. 63/094,498 filed on Oct. 21, 2020, the disclosure of each of which is incorporated herein, in their entirety, by this reference.

### BACKGROUND

In various circumstances, a person may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Additionally, bed pans, which are receptacles used for the toileting of bedridden patients, such as those in a health care facility, are sometimes used. Bed pans, however, can be prone to discomfort, spills, and other hygiene issues.

Females who suffer the most severe consequences of urinary incontinence, such as discomfort, rashes, and sores are typically elderly and often bedbound. They also require continuous assistance to maintain hygiene. These patients typically lay on their back for extended periods of time and often have difficulty reaching and manipulating urine collection devices.

### SUMMARY

Embodiments are directed to fluid collection devices suitable for collecting and transporting fluid, such as urine, away from the body of a person. In an embodiment of the present disclosure, a fluid collection device is disclosed. The fluid collection device includes a casing which defines an internal volume and an opening. The casing can include or otherwise be formed of a compressible material which is elastically deformable when the fluid collection device is disposed in a pubic region of a female user. The fluid collection device includes a fluid permeable material disposed within the internal volume and in fluid communication with the opening.

In another embodiment of the present disclosure, a fluid collection device is disclosed. The fluid collection device includes a fluid impermeable housing defining an internal volume and an opening. The device includes a casing substantially overlaying the fluid impermeable housing. The device includes a fluid permeable material disposed within the internal volume and in fluid communication with the opening. The casing can be formed of a compressible material elastically deformable when the fluid collection device is disposed in a pubic region of a female user.

In another embodiment of the present disclosure, a system for transporting urine away from a user is disclosed. The system includes a fluid collection device and a suction device. The suction device is operably coupled to the fluid collection device such that fluid can be drawn from the fluid collection device via the suction device. The fluid collection device includes a casing which defines an internal volume and an opening. The casing can include or otherwise be formed of a compressible material which is elastically deformable when the fluid collection device is disposed in a pubic region of a female user. The fluid collection device includes a porous material disposed within the internal volume and in fluid communication with the opening.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a urine collection device, according to an embodiment.

FIG. 1B is a top plan view of the urine collection device shown in FIG. 1A.

FIG. 2A is a schematic cross-section view of the urine collection device taken through the section lines 2A-2A of FIG. 1A.

FIG. 2B is a schematic cross-section view of the urine collection device taken through the section lines 2B-2B of FIG. 1B.

FIG. 3A is a top plan view of the urine collection device in an uncompressed state.

FIG. 3B is a top plan view of the urine collection device in a compressed state.

FIG. 4A is an isometric view of a urine collection device, according to an embodiment.

FIG. 4B is a schematic cross-section view of the urine collection device taken through the section lines 4B-4B of FIG. 4A.

FIG. 5 is a block diagram of a system for urine collection, according to an embodiment.

### DETAILED DESCRIPTION

Embodiments are directed to urine collection devices suitable for collecting and transporting urine away from the body of a person. The embodiments of the urine collection devices described herein may be positioned in a pubic region of a female user to collect urine or other fluids expelled by the user. While this disclosure primarily describes the device as a urine collection device, it will be appreciated that the embodiments disclosed herein can also be utilized to collect other fluids discharged from a female user. In embodiments, the urine collection device includes a casing forming or defining an interior volume and an opening to the interior volume. The casing includes a compressible material that is elastically deformable when the fluid collection device is disposed in the pubic region of the female user. The urine collection device includes a porous or fluid permeable material disposed within the internal volume and in fluid communication with the opening.

The urine collection device can include a fluid conduit disposed within the internal volume and in fluid communication with at least a portion of the interior volume. The fluid conduit can enable extraction of fluid from the internal volume. For example, the fluid conduit can be coupled to a vacuum source which extracts urine or other fluids from the internal volume by applying a negative pressure to the internal volume of the receptacle. One suitable non-limiting example of a vacuum source that can be used is the DryDoc Vacuum Station, available from Pure Wick, Inc.

In embodiments, the casing can include and/or house a plurality of layers, for example, one or more fluid permeable layers, one or more absorbent layers, one or more hydrophobic layers, one or more fluid permeable layers, one or more fluid impermeable layers, and so on. For example, a porous material can be positioned within the interior volume to provide an interface with the user's genitals that is substantially moisture free. Additionally, or alternatively, the fluid permeable layer can have hydrophobic attributes that cause the fluid to flow to a fluid collection region within the casing.

In use, the urine collection device can be positioned in a pubic region of a female user. For example, the urine collection device can be disposed between the thighs and/or buttocks of the female user and abut a urethral opening of the female user's genitals. A portion of the urine collection device can abut or otherwise be disposed adjacent a perineum of the female user. While positioned in the pubic region of the female user, the compressible material of the casing can be compressed, elastically deformed, or otherwise manipulated to conform to the pubic region of the female user to more proficiently retain the urine collection device in a fixed position relative to the user. By incorporating a casing made up of a compressible material and having a relatively large diameter (e.g., between about 2 cm and about 10 cm), the urine collection device can be reliably retained by even small persons having relatively small thighs and buttocks.

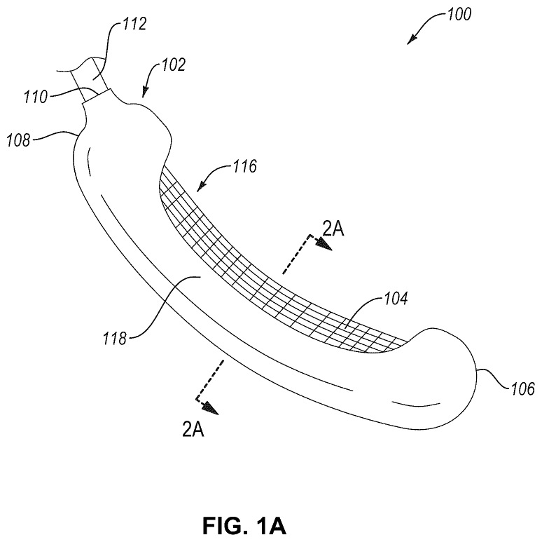

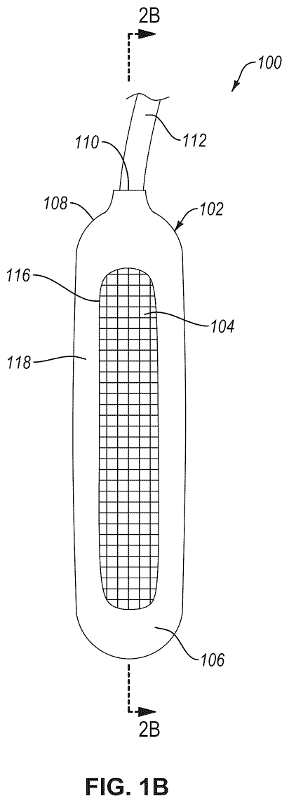

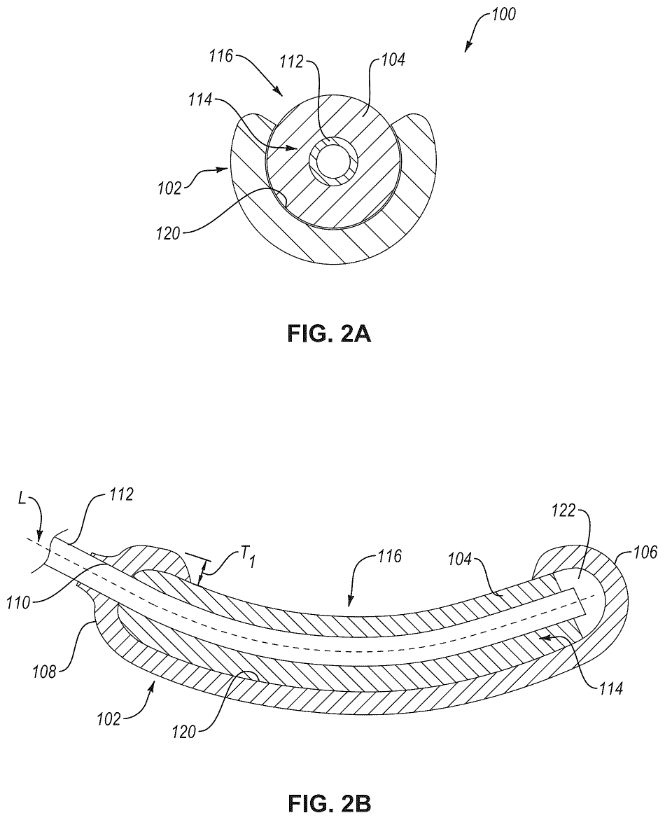

FIGS. 1A-1B are respective isometric and top plan views of a urine collection device 100, according to an embodiment. The urine collection device 100 includes a casing 102 and a fluid permeable layer or porous material 104 disposed within the casing 102. In some embodiments, the casing 102 can be defined as having an elongated cylindrical shape with a radius of curvature such that a longitudinal axis L of the casing 102 bends along the length of the longitudinal axis L (see FIG. 2B) to better fit to the subject's anatomy. The casing 102 can include a distal end 106 and a proximal end 108. The distal end 106 can be closed as to form a fluid collection region within the casing 102. The proximal end 108 can form an aperture 110 wherein a fluid conduit 112 can be partially disposed within the casing 102.

The casing 102 can form or otherwise define an interior cavity or internal volume 114 within the casing 102 (see FIGS. 2A and 2B). The casing 102 can define an opening 116 in fluid communication with the internal volume 114. Urine and other fluids expelled by the user can be received within the opening 116 while the urine collection device 100 is positioned at a pubic region of the user. In embodiments, the opening 116 can be elongated and extend longitudinally across a surface 118 of the casing 102. Although the embodiment depicted in FIGS. 1A and 1B illustrate a single elongated opening 116, other embodiments may include two or more openings of various geometric shapes and sizes. For example, the casing may form two or more rectangular shaped openings, each in fluid communication with the internal volume of the casing, according to an embodiment.

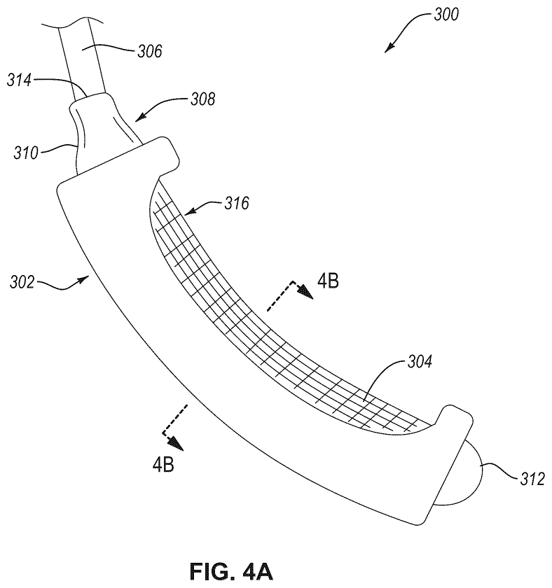

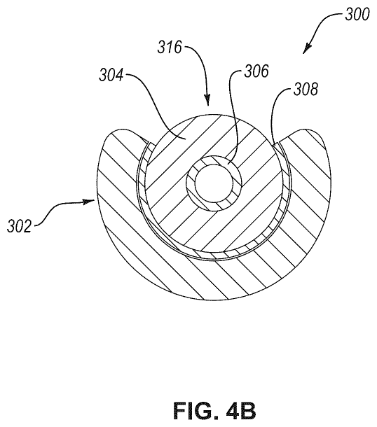

The casing 102 can be manufactured or formed out of a compressible material, such as, a compressible foam that is elastically deformable when the fluid collection device 100 is disposed in the pubic region of the female user. The compressible foam can be hydrophobic or otherwise fluid impermeable such that the casing 102 does not absorb liquid, nor is liquid able to leak out of the casing 102. In embodiments, the casing 102 can be a formed of a closed cell and fluid impermeable foam. As will be discussed in relation to FIGS. 4A and 4B, the casing can be formed of a compressible material which is adhered to and overlays at least a portion of a fluid impermeable housing that defines the internal volume of the urine collection device, in some embodiments.

The porous material 104 can be disposed partially or entirely within the internal volume 114 formed within the casing 102. For example, a majority of the porous material 104 can be disposed within the internal volume 114, while a minority of the porous material 104 can extend into and, sometimes, out of the opening 116 to contact or nearly contact a urethral opening of the female patient. In embodiments, a portion of the porous material 104 is exposed via the opening 116 of the casing 102. The exposed porous material 104 can function as a fluid conduit which wicks fluid away from the user and into the internal volume 114.

In embodiments, the porous material 104 can include multiple layers of material, such as, hydrophobic or fluid permeable layers having varied permeable properties. The porous material 104 can include any material that can wick, draw, or direct fluid, for example, the porous material 104 can include fabric, such as a gauze (e.g., a silk, linen, polyester, or cotton gauze), another soft fabric (e.g., jersey knit fabric or the like), or another smooth fabric (e.g., rayon, satin, or the like). In some examples, the porous material 104 can include an open cell foam. Forming the porous material 104 from gauze, soft fabric, and/or smooth fabric can reduce chafing caused by the urine collection device 100. The porous material 104 can additionally or alternatively be formed using one or more layers of polytetrafluoroethylene (PTFE) and/or spun plastic.

The porous material 104 can include permeable material designed to wick or pass fluid therethrough. The permeable properties referred to herein may be wicking, capillary action, diffusion, or other similar properties or processes, and are referred to herein as “permeable” and/or “wicking.” Such “wicking” may not include absorption of fluid into the porous material 104. Put another way, substantially no absorption of fluid into the porous material 104 may take place after the porous material 104 is exposed to the fluid and removed from the fluid for a time. While no absorption is desired, the term “substantially no absorption” may allow for nominal amounts of absorption of fluid into the wicking material (e.g., absorbency), such as less than about 30 wt % of the dry weight of the porous material 104, less than about 20 wt %, less than about 10 wt %, less than about 7 wt %, less than about 3 wt %, less than about 2 wt %, less than about 1 wt %, or less than about 0.5 wt % of the dry weight of the porous material 104. The porous material 104 can include natural fibers. In such examples, the porous material 104 may have a coating to prevent or limit absorption of fluid into the material, such as a water repellent coating.

In embodiments, the porous material 104 can have a high absorptive rate and a high permeation rate such that urine can be rapidly absorbed and/or transported through the porous material 104. In some embodiments, the porous material 104 can include and/or have the moisture-wicking characteristic of gauze, felt, terrycloth, thick tissue paper, and/or a paper towel. For example, the porous material 104 can be formed from spun nylon fibers with an exterior layer of gauze or other suitable fabric that contacts the subject. In some embodiments, the porous material 104 can be soft and/or minimally abrasive such that the porous material 104 does not irritate the skin of the user. The porous material 104 can wick, draw, or direct fluid away from the skin of the user such that moisture at or near the skin is lessened. In some embodiments, the porous material 104 can have moisture-wicking properties. The moisture-wicking properties of the porous material 104 can help prevent urine from leaking onto, for example, a hospital bed or a user of the urine collection device 100. In some embodiments, the porous material 104 can be formed of fine denier polyester fibers coated with a thermoplastic water-based binder system.

The fluid conduit 112 can be formed as an elongate tube which extends from the internal volume 114 of the casing 102 to outside of the casing 102. For example, the fluid conduit 112 can span substantially parallel to the longitudinal axis L of the casing 102 and extend through the aperture 110 within the casing 102. When suction is applied to the fluid conduit 112, the fluids disposed within the casing 102 can be drawn through the fluid conduit 112 and out of the urine collection device 100.

FIG. 2A is a section view of the urine collection device 100 taken through the section lines 2A of FIG. 1A, according to an embodiment. In embodiments, the porous material 104 and the fluid conduit 112 can substantially fill the internal volume 114 of the casing 102. In some embodiments, the combination of the porous material 104 and fluid conduit 112 may only occupy a portion of the internal volume 114 of the casing 102. In embodiments, the porous material 104 can be wrapped or otherwise positioned around a periphery of the portion of the fluid conduit 112 positioned within the internal volume 114 of the casing 102. In some embodiments, the portion of the fluid conduit 112 positioned within the internal volume 114 can be enclosed by the porous material 104. The porous material 104 can be adhered, welded, otherwise affixed to an inner surface 120 of the casing 102 and/or the fluid conduit 112, or merely disposed in the casing 102 for a resilient and tight fit therein.

As shown in FIG. 2A, a portion of the porous material 104 can protrude or otherwise extends through the opening 116. This portion of the porous material 104 can interface part of the pubic region of the female user. For example, the portion of the porous material 104 that protrudes through the opening can interface with the urethral opening of the female user. However, in other embodiments, the portion of the porous material 104 that extends through the opening 116 may not directly contact the female user, in some embodiments. Rather, a gap may exist between the female user and the portion of the porous material 104 that extends through the opening 116.

FIG. 2B is a section view of the urine collection device 100 taken through the section lines 2B of FIG. 1B, according to an embodiment. As shown in the embodiment of the urine collection device 100 illustrated in FIG. 2B, the urine collection device 100 can define a longitudinal axis L extending longitudinally from the distal end 106 of the casing 102 to the proximal end 108 of the casing 102. The fluid conduit 112 can be disposed along the longitudinal axis L of the urine collection device 100. Additionally, or alternatively, the fluid conduit 112 can be offset from the longitudinal axis L yet extend substantially parallel to the longitudinal axis L of the urine collection device 100.

The casing 102 can define a collection region 122 (e.g., a reservoir) that accumulates fluid expelled within the urine collection device 100, for example, gravitational forces can direct fluid within the internal volume 114 to accumulate at the collection region 122. In other words, the collection region 122 can temporarily store a quantity of fluid within the urine capture device 100 prior to extraction of the fluid using the fluid conduit 112. A portion of the fluid conduit 112 can be in fluid communication with the collection region 122 such that fluid accumulated at the collection region 122 can be extracted from the urine collection device 100 via the fluid conduit 112 (e.g., by applying a negative pressure to the fluid conduit using a vacuum source). In some embodiments, the collection region 122 may be substantially free of the porous material 104 as to provide substantially all of the volume of the collection region 122 to accumulate fluids.

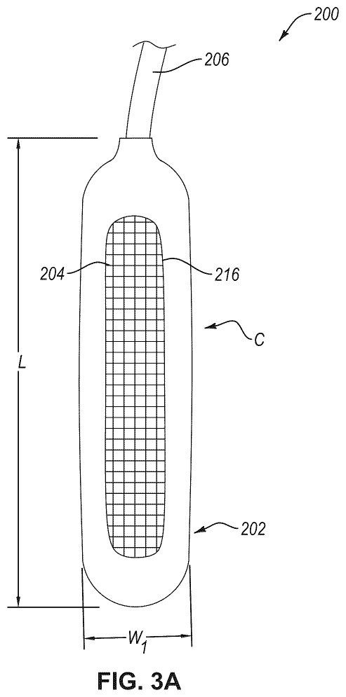

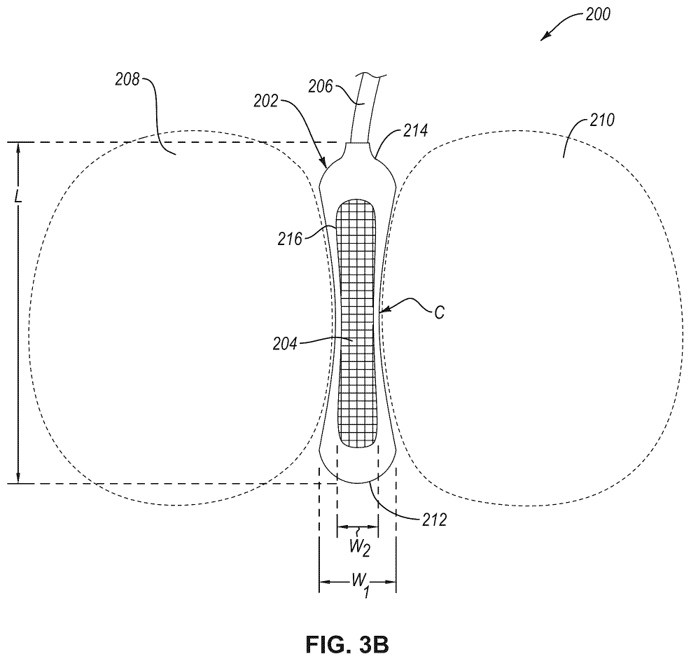

As shown in the embodiment of the urine collection device 100 illustrated in FIG. 2B, the casing 102 has a substantially uniform thickness T₁ of compressible material forming the internal volume 114. In embodiments, the thickness T₁ of the casing 102 can be at least 3 millimeters, about 3 millimeters to about 25 millimeters, about 3 millimeters to about 6 millimeters, about 6 millimeters to about 9 millimeters, about 9 millimeters to about 12 millimeters to about 15 millimeters, about 15 millimeters to about 18 millimeters, about 18 millimeters to about 21 millimeters, about 21 millimeters to about 25 millimeters, or less than 25 millimeters. In embodiments, the casing 102 can have a thickness which varies along a length of the casing 102. For example, the thickness of the casing 102 can be greater at the proximal and distal ends 106, 108 than a thickness of the casing 102 at a central region (e.g., central region C shown in FIGS. 3A and 3B).

FIGS. 3A and 3B depict an embodiment of a urine collection device 200 including a casing 202, porous material 204, and a fluid conduit 206. More particularly, FIG. 3A shows the urine collection device 200 prior to be disposed in a pubic region of a female user while FIG. 3B shows the urine collection device 200 disposed in the pubic region of the female user. The casing 202 can define a substantially constant width W₁ through a length L of the urine collection device 200 prior to being disposed in the pubic region of a female user. Once disposed in the pubic region of the female user, however, the casing 202 can be compressed or elastically deformed to conform to the pubic region of the female user to more proficiently retain the urine collection device 200 in a fixed position relative to the female user. While positioned in the pubic region (e.g., between the thighs 208, 210 of the female user), the casing 202 can be compressed or otherwise deformed such that a width of the casing 202 varies along the length of the casing 202. For example, as shown in FIG. 3B, the width W₁ at a distal end 212 and a proximal end 214 of the casing 202 can be unaltered while a width W₂ at a central region C of the casing 202 can be narrower due to compressive forces exerted on the central region of the casing 202 by the user's thighs 208, 210.

While FIG. 3B shows the central region C as being compressed and the distal and proximal ends 212, 214 as being substantially unaffected, one or more of the central region C and the distal and proximal ends 212, 214 can be compressed or otherwise deformed while the urine collection device 200 is disposed in the pubic region of the user. For example, one or both of the distal and proximal ends 212, 214 can be compressed or deformed while the central region C is substantially unaffected. Moreover, while the user's thighs 208, 210 are depicted as compressing or deforming the casing 202, other portions of the user's body can alternatively or additionally compress or deform the casing 202. For example, the user's labia and/or buttocks may also contact the urine collection device 200 to compress or deform the casing 202. In embodiments, an opening 216 formed within the casing 202 may not be deformed or altered when the casing 202 is compressed or deformed by the pubic region of the user.

FIG. 4A is an isometric view of a urine collection device 300, according to an embodiment. The urine collection device 300 includes a casing 302, a porous material 304, a fluid conduit 306, and a fluid impermeable housing 308. The fluid impermeable housing 308 can define an internal volume or cavity within which the porous material 304 can be at least partially disposed. The porous material 304 can have the features and functionality of other porous or fluid permeable material described herein, for example, porous material 104, 204. The casing 302 can be separate and distinct from the fluid impermeable housing 308, which is coupled or mounted to the fluid impermeable housing 308. The casing 302 can be disposed over at least a portion of the fluid impermeable housing 308. For example, the casing 302 can overlay a majority of the fluid impermeable housing 308 except for a proximal end 310 and a distal end 312 of the fluid impermeable housing 308, as shown in FIG. 4A. The proximal end 310 of the fluid impermeable housing 308 can form an opening 314 which receives or retains the fluid conduit 306. The fluid conduit 306 include features and functionality as other fluid conduits described herein, for example, fluid conduit 112. The casing 302 and the fluid impermeable housing 308 can form or define an opening 316 in fluid communication with the internal volume. Urine and/or other fluids expelled by the user can be received within the opening 316 while the urine collection device 300 is positioned at a pubic region of the user. In embodiments, the opening 316 can be elongated and extend longitudinally across along the urine collection device 300.

The fluid impermeable housing 308 can be formed of any suitable fluid impermeable materials, such as a fluid impermeable polymer (e.g., silicone, polypropylene, polyethylene, polyethylene terephthalate, polyurethane, a polycarbonate, polyvinyl chloride, latex, silicone, etc.), a metal or alloy layer or film, another suitable material, or combinations thereof. In some embodiments, the casing 302 can be formed of any suitable fluid impermeable materials, such as a fluid impermeable polymer (e.g., silicone, polypropylene, polyethylene, polyethylene terephthalate, polyurethane, a polycarbonate, polyvinyl chloride, latex, silicone, etc.), a metal or alloy layer or film, another suitable material, or combinations thereof.

In other embodiments, the casing 302 need not be fluid impermeable because the fluid impermeable housing 308 may sufficiently prevent fluid from leaking or seeping from the urine collections device 300. In these embodiments, the casing 302 can include any material that can be compressed, elastically deformed, or otherwise manipulated to conform to the pubic region of the female user to more proficiently retain the urine collection device 300 in a fixed position relative to the user. For example, the casing 302 can be manufactured from an open cell foam, such as, a hydrophobic polyurethane foam.

The porous material 304 can include any material that can wick, draw or direct fluid, for example, the porous material 304 can include fabric, such as a gauze (e.g., a silk, linen, polyester, or cotton gauze), another soft fabric (e.g., jersey knit fabric or the like), another smooth fabric (e.g., rayon, satin, or the like). In some examples, the porous material 304 can include an open cell foam, spun nylon fibers, or other suitable synthetic porous material.

In embodiments, the casing 302 can be adhered, molded, welded, printed, or otherwise affixed to overlay at least a portion of the fluid impermeable housing 308. The casing 302 can be manufactured or formed out of a compressible material, such as, a compressible foam that is elastically deformable when the fluid collection device 300 is disposed in the pubic region of the female user. The compressible foam can be hydrophobic or otherwise fluid impermeable such that the casing 302 does not absorb liquid, nor is liquid able to leak out of the casing 302. In embodiments, the casing 102 can be a formed of a closed cell and fluid impermeable foam. While positioned in the pubic region of the female user, the casing 302 can be compressed, elastically deformed, or otherwise manipulated to conform to the pubic region of the female user to more proficiently retain the urine collection device in a fixed position relative to the user. By incorporating a casing 302 made up of a compressible material and having a relatively large diameter (e.g., between 2 cm and 10 cm), the urine collection device 300 can be reliably retained by even small persons having relatively small thighs and buttocks.

FIG. 4B is a section view of the urine collection device 300 taken through the section lines 4B of FIG. 4A, according to an embodiment. The casing 302 and the fluid impermeable housing 308 can define the opening 316 and the porous material 304 and/or the fluid conduit 306 can be at least partially disposed within the internal cavity or volume formed by the fluid impermeable housing 308. As shown in FIG. 4B, the casing 302 can be overlaid onto at least a portion of the fluid impermeable housing 308 such that the casing 302 surrounds a portion of the fluid impermeable housing 308.

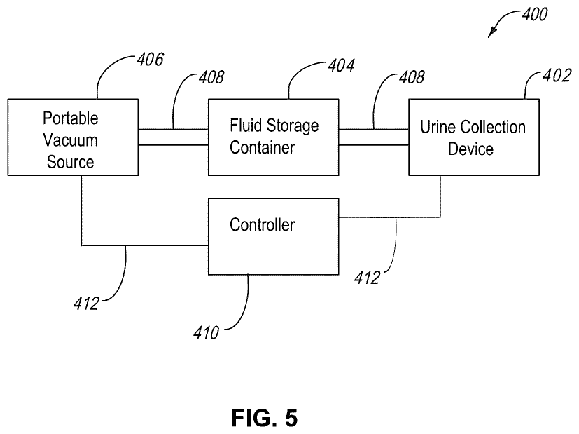

FIG. 5 is a block diagram of a system 400 for fluid collection (e.g., transporting urine away from a user), according to an embodiment. The system 400 includes a urine collection device 402, a fluid storage container 404, a portable vacuum source 406, one or more fluid conduits 408, and a controller 410. The urine collection device 402 may include any of the urine collection devices described herein, such as the urine collection device 100, 200, 300. The urine collection device 402, the fluid storage container 404, and the portable vacuum source 406 may be fluidly coupled to each other via one or more fluid conduits 408. The one or more fluid conduits 408 may include tubes, such as the fluid conduit 112 previously described herein. The urine collection device 402 may be operably coupled to one or more of the fluid storage container 404 or the portable vacuum source 406 via the fluid conduit 408. Urine and/or other bodily fluids collected in the urine collection device 402 may be removed from the urine collection device 402 via the fluid conduit 408. For example, a first open end of the fluid conduit 408 may be coupled to the urine collection device 402. The second open end of the fluid conduit 408 may be coupled to the portable vacuum source 406. A suction force may be introduced into an interior volume of the urine collection device 402 via the first open end of the fluid conduit 408 responsive to a suction (e.g., vacuum) force applied at the second end of the fluid conduit 408. The suction force may be applied to the second open end of the conduit 408 by the portable vacuum source 406 either directly or indirectly.

The suction force may be applied indirectly via the fluid storage container 404. For example, the second open end of the fluid conduit 408 may be disposed within the fluid storage container 404 and an additional fluid conduit 408 may extend from the fluid storage container 404 to the portable vacuum source 406. Accordingly, the portable vacuum source 406 may apply suction to the urine collection device 402 via the fluid storage container 404. The suction force may be applied directly via the fluid storage container 404. For example, the second open end of the fluid conduit 408 may be disposed within the portable vacuum source 406. An additional fluid conduit 408 may extend from the portable vacuum source 406 to a point outside of the urine collection device 402, such as to the fluid storage container 404. In such examples, the portable vacuum source 406 may be disposed between the urine collection device 402 and the fluid storage container 404.

In some embodiments, the fluid storage container 404 may include a bag (e.g., drainage bag), a bottle or cup (e.g., collection jar), or any other enclosed container for storing bodily fluids such as urine. In examples, the fluid conduit 408 may extend from the urine collection device 402 and attach to the fluid storage container 404 at a first point therein. An additional fluid conduit 408 may attach to the fluid storage container 404 at a second point thereon and may extend and attach to the portable vacuum source 406. For example, the fluid storage container 404 may include a container fluidly coupled to a first fluid conduit 408 that is also fluidly coupled to the outlet of the urine collection device 402. The container may be fluidly coupled to a second fluid conduit 408 that is also fluidly coupled to a portable vacuum source 406. In such examples, the portable vacuum source 406 may provide a vacuum/suction through the container to the outlet to provide suction in the interior volume of the urine collection device. Accordingly, a vacuum (e.g., suction) may be drawn through the urine collection device 402 via the fluid storage container 404. As the fluid (e.g., urine) is drained from the chamber, the fluid may travel through the first section of conduit to the fluid storage container where it may be retained. Fluid, such as urine, may be drained from the urine collection device 402 using the portable vacuum source 406.

The portable vacuum source 406 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 406 may provide a vacuum or suction to remove fluid from the outlet of the urine collection device 402. In some embodiments, the portable vacuum source 406 may be powered by one or more of a power cord (e.g., connected to a power socket), one or more batteries, or even manual power (e.g., a hand operated vacuum pump). In examples, the portable vacuum source 406 may be sized and shaped to fit outside of, on, or within the urine collection device 402. For example, the portable vacuum source 406 may include one or more miniaturized pumps or one or more micro pumps. The portable vacuum sources 406 disclosed herein may include one or more of a switch, a button, a plug, a remote, or any other device suitable to activate the portable vacuum source 406. It should be understood that the portable vacuum sources 406 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

The controller 410 can be communicatively coupled (via electrical connections 412 or wirelessly) to one or more of the urine collection device 402 or the portable vacuum source 406. The controller 410 can direct the portable vacuum source 406 to operate when a moisture event is detected. In any of the examples, systems, or devices disclosed herein, the urine collection device 402 may include moisture sensors (not shown) disposed inside of the urine collection device 402. In such examples, the moisture sensor may be operably coupled to the controller 410 or directly to the portable vacuum source 406, and may provide electrical signals indicating that moisture is or is not detected within the urine collection device 402. The moisture sensor(s) may provide an indication that moisture is present, and responsive thereto, the controller 410 or portable vacuum source 406 may direct the initiation of suction to the outlet to remove the fluid from the urine collection device 402. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the urine collection device 402 and may provide a signal to the controller 410 and/or portable vacuum source 406 to activate the portable vacuum source 406. In other embodiments, the controller 410 may direct the portable vacuum source 406 to continuously operate.

While various embodiments of the urine collection system, methods and devices have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

1. A fluid collection device, comprising:
a compressible component defining an internal volume;
a fluid impermeable housing disposed at least partially within the internal volume of the compressible component and defining a cavity, a region of the fluid impermeable housing at least partially defining an opening, wherein the compressible component is positioned adjacent to at least a portion of the region of the fluid impermeable housing defining the opening; and
a fluid permeable material disposed at least partially within the cavity of the fluid impermeable housing and extending at least partially across the opening.

2. The fluid collection device of claim 1, wherein the compressible component includes a compressible material that is elastically deformable when the fluid collection device is disposed in a pubic region of a female user.

3. The fluid collection device of claim 1, wherein the compressible component is sized and configured with a thickness of the compressible component that varies along a length of the compressible component when a portion of the compressible component is compressed between the thighs and/or buttocks of the female user.

4. The fluid collection device of claim 2, wherein the compressible component includes a compressible material including a foam that is elastically deformable, the compressible component having a thickness of at least about 3 mm.

5. The fluid collection device of claim 3, wherein the compressible component has a thickness of about 6 mm to about 9 mm and a diameter of about 2 cm to about 10 cm.

6. The fluid collection device of claim 1, wherein the compressible component includes a closed-cell foam, and wherein the compressible component and the fluid impermeable housing are substantially cylindrical.

7. The fluid collection device of claim 1, further comprising a fluid conduit disposed within the cavity and configured to enable extraction of fluid from the cavity, wherein the fluid impermeable housing includes a proximal end region having the fluid conduit extending therethrough and a distal end region, at least one of the proximal end region or the distal end region remaining uncovered by the compressible component.

8. The fluid collection device of claim 1, wherein the fluid impermeable housing is separate and distinct from the compressible component and the internal volume of the compressible component is shaped substantially complementary to the fluid impermeable housing.

9. The fluid collection device of claim 1, wherein the fluid impermeable housing is adhesively bonded to the compressible component, the compressible component substantially overlaying the fluid impermeable housing.

10. The fluid collection device of claim 1, wherein the fluid impermeable housing is at least partially positioned between the compressible component and the fluid permeable material, at least a portion of the fluid permeable material protruding from within the cavity outward away from the compressible component and the fluid impermeable housing, and through the opening at least partially defined by the fluid impermeable housing and the compressible component.

11. The fluid collection device of claim 7, wherein:
the fluid permeable material is disposed within the cavity adjacent to the fluid impermeable housing; and
the fluid permeable material protrudes from within the cavity outward away from the compressible component and beyond at least a portion of the longitudinally extending edges of the compressible component and the fluid permeable housing defining the opening.

12. The fluid collection device of claim 1, wherein the compressible component and the fluid impermeable housing include complementary edges positioned adjacent and substantially aligned with one another and at least partially defining the opening such that the opening, the compressible component covering at least a portion of the region of the fluid impermeable housing defining the opening.

13. A fluid collection device, comprising:
a fluid impermeable housing having a proximal end region and a distal end region, the fluid impermeable housing defining an internal volume and at least partially defining an opening of the fluid collection device;
a fluid permeable material disposed within the internal volume of the fluid impermeable housing and extending across at least a portion of the opening; and
a compressible component having at least a portion that is elastically deformable when the fluid collection device is disposed in a pubic region of a female user,
wherein the fluid impermeable housing is at least partially positioned within the component and at least one of the proximal end region or the distal end region of the fluid impermeable housing are not covered by the component.

14. The fluid collection device of claim 13, wherein the compressible component is separate and distinct from the fluid impermeable housing and substantially overlaying the fluid impermeable housing, the component.

15. The fluid collection device of claim 13, wherein the compressible component has a thickness of about 3 mm to about 25 mm and includes a compressible material.

16. The fluid collection device of claim 13, wherein the compressible component has a thickness that varies along a length of the casing.

17. The fluid collection device of claim 13, wherein the compressible component is a hydrophobic polyurethane open cell foam.

18. The fluid collection device of claim 13, wherein the fluid impermeable housing is at least partially positioned between the component and the fluid permeable material, at least a portion of the fluid permeable material protruding from within the cavity outward away from the component and the fluid impermeable housing, and through the opening at least partially defined by the compressible component and the fluid impermeable housing.

19. The fluid collection device of claim 13, wherein a region of the fluid impermeable housing partially defines the opening and wherein the internal volume of the compressible component is shaped substantially complementary to the fluid impermeable housing such that the compressible component covers at least a portion of the region of the fluid impermeable housing defining the opening.

20. A system for transporting urine away from a female user, the system comprising:
a fluid collection device, comprising:
a fluid impermeable housing defining a cavity, a region of the fluid impermeable housing at least partially defining an opening in the fluid collection device, wherein the cavity is configured to collect fluid discharged into the fluid impermeable housing through the opening;
a compressible component positioned adjacent to at least a portion of the region of the fluid impermeable housing defining the opening; and
a porous material disposed at least partially within the cavity of the fluid impermeable housing and extending at least partially across the opening; and
a suction device operably coupled to the fluid collection device, the suction device configured to draw the fluid collected in the cavity fluid collection device.
